(19) United States
(12) Patent Application Publication
(10) Pub. No.: US 2026/0354790 A1
(43) Pub. Date: Nov. 12, 2026

(54) METHOD AND APPARATUS FOR ASSESSING HEALTH

(71) Applicant: Wayne State University, Detroit, MI (US)

(72) Inventors: Charles S. Chung, Detroit, MI (US); Lindsay A. Pittman, Edwardsburg, MI (US); Michelle L. Milne, Lexington Park, MD (US)

(21) Appl. No.: 19/120,834

(22) PCT Filed: Oct. 13, 2023
(86) PCT No.: PCT/US2023/035074

§ 371 Date: Apr. 14, 2025

Related U.S. Application Data
(60) Provisional application No. 63/416,180, filed on Oct. 14, 2022.

(51) Int. Cl. A61B 8/08; A61B 8/00

(52) U.S. Cl. CPC A61B 8/0858; A61B 8/5223

(57) ABSTRACT

A compute system that stores a first quantity associated with ultrasonic backscatter arising from a first region of an organ at a first angle of insonification during a first session. The compute system also stores a second quantity associated with ultrasonic backscatter arising from a second region of the organ at a second angle of insonification during the first session. Further, the compute system determines a first difference between the first quantity and the second quantity provides a health characteristic of the organ to a user interface, wherein the health characteristic is based on the first difference.

### CROSS-REFERENCE TO RELATED APPLICATION

This application claims priority to U.S. Provisional Application 63/416,180, filed Oct. 14, 2022, the contents of which is incorporated herein by reference in its entirety.

### GOVERNMENT LICENSE RIGHTS

This invention was made with government support under Grant No. 1R01HL15738 awarded by the National Institutes of Health-National Heart, Lung, and Blood Institute, and under Grant No. R01EB030058 awarded by the National Institute of Biomedical Imaging and Bioengineering. The government has certain rights in the invention.

### FIELD OF TECHNOLOGY

Exemplary fields of technology for the present disclosure may relate to the health assessment of an organ or tissue.

### BACKGROUND

In a healthy myocardium, ventricular function is generally optimized by myofibers that are orientated helically and circumferentially. Changes in myocardial structure, myofiber alignment, collagen deposition, and collagen structure may be indicative of cardiomyopathies as well as systemic disease and may result in a loss of cardiac function. The extracellular matrix (ECM) myocardial fibrosis is considered to be the dysregulation of degradation and deposition of ECM proteins, including collagen, which lies throughout the myocardium and bundles groups of cardiomyocytes together into myofibers. Collagen also forms hydroxyproline crosslinks between fibrils that stiffen the myocardium, increase the tensile strength of the extracellular matrix, and increases the matrix resistance to degradation by many proteases, besides specific collagenases. Although myocardial fibrosis is recognized as a pathological process following myocardial injury and as an indicator of cardiomyopathies and systemic disease, protocols for monitoring myocardial fibrosis disease progression are generally absent in current clinical practice.

Issues with collagen, and collagen cross-linking, may also pose problems with other organs or tissues.

Current clinical protocols to investigate cardiac structure typically utilize ventricular biopsy or magnetic resonance imaging (MRI), which may be invasive, costly, and/or contraindicated.

As such, there is a need for systems and methods to non-invasively assess the health of tissues and/or organs that may suffer from collagen cross-linking.

### BRIEF DESCRIPTION OF THE DRAWINGS

FIGS. 1A-1B illustrate an exemplary system for assessing health or an organ or tissue;

FIGS. 1C and 1D illustrate interactions between collagen alignment and incoming ultrasonic waves;

FIG. 2 illustrates another exemplary system for assessing health or an organ or tissue;

FIGS. 3A and 3B illustrate another exemplary system for assessing health or an organ or tissue;

FIG. 4 illustrates an exemplary graph that may be employed to assess health of an organ or tissue over time;

FIG. 5 illustrates an exemplary table that may be employed to assess health of an organ or tissue;

FIG. 6 illustrates an exemplary technique for assessing health of an organ or tissue;

FIG. 7 illustrates an exemplary technique for assessing health of an organ or tissue over time; and

FIG. 8 illustrates an exemplary compute system that may be employed in systems and techniques described herein.

### DETAILED DESCRIPTION

The examples, techniques, and systems set forth herein employ ultrasonic backscatter to assess health of an organ or tissue. The organs or tissue discussed herein may refer to human organs or tissues, or to other non-human organs or tissues.

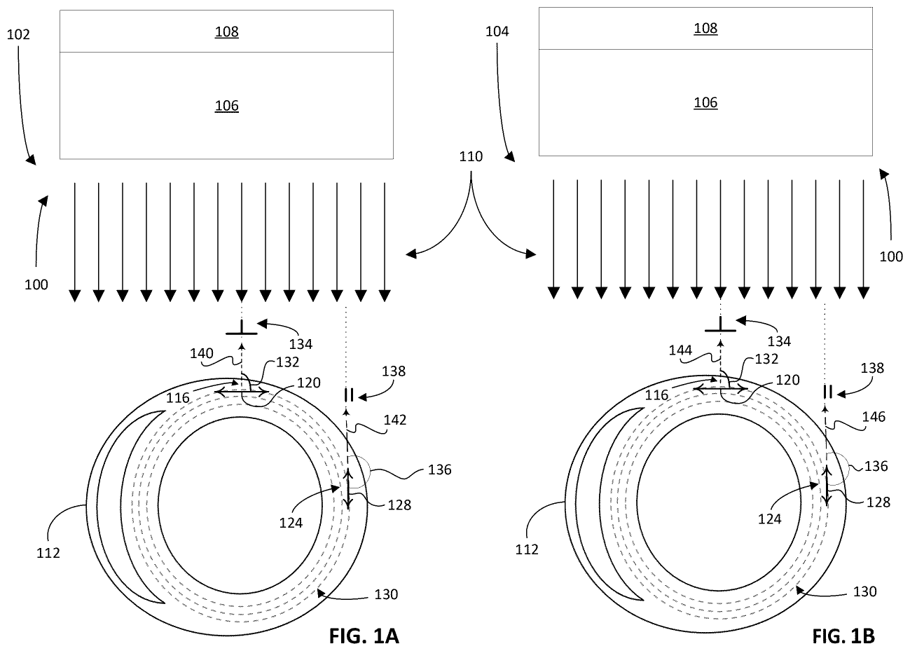

With reference to FIGS. 1A-1B, an exemplary system 100 in operation is represented during a first session 102 and a second session 104 is shown. The exemplary system 100 includes an exemplary ultrasonic transducer 106 coupled to an exemplary compute system 108. The ultrasonic transducer 106 conveys ultrasonic waves 110 toward an organ or tissue 112 (exemplary heart shown). In addition to conveying ultrasonic waves 110 toward the organ or tissue 112, the transducer 106 is configured to also receive ultrasonic backscatter from the organ or tissue 112. For sake of discussion the organ or tissue 112 will simply be rereferred to as organ 112, though it is understood that organ 112 could be substituted by tissue 112 or some combination of tissue and organ.

Differing regions of the organ 112 backscatter (i.e., reflect) differing amounts of ultrasonic waves. The compute system 108 stores data related to the backscatter received by the ultrasonic transducer 106 (e.g., a linear array transducer). This data may, for example, be employed to create an image of the organ 112. It is noted, however, that not all systems 100 need to produce images. For example, some transducers, such as a single crystal transducer, merely allows for the storage of data associated with differing amounts of backscatter received from differing regions and need not produce an image.

Where images are produced, it is understood that brighter regions in the image generally represent a higher quantity of backscatter than in dimmer regions of the same organ. This variation in brightness is a hallmark of anisotropy in the backscatter from the organ. Similarly, in systems where images are not produced, variation in backscatter quantity is a hallmark of anisotropy in the backscatter from the organ.

The brightest regions (or the data that represents the greatest amount of backscatter associated with corresponding region or location data) are generally regions where the average collagen alignment direction is perpendicular to the incoming ultrasonic waves 110. Conversely, dimmer regions (or the data that represents a lower amount of backscatter) generally represent lower quantities of backscatter received from the associated region(s) of the organ. Further, there may be regions where the average collagen alignment direction in that region is parallel to the impinging ultrasonic waves. These regions often produce the lowest amount of backscatter, if any. Accordingly, as the angle of insonification (i.e., the angle at which an ultrasonic wave impinges the average collagen alignment direction in a particular region) moves from ninety to one hundred eighty degrees, the resulting backscatter generally decreases.

Before proceeding with further discussion of the system 100 of FIGS. 1A and 1B, reference is made to FIGS. 1C and 1D to illustrate average collagen alignment direction in different regions of an organ or tissue. In FIG. 1C, an exemplary representation 114 of ultrasonic waves 110 impinging a first region 116 having a first set of collagen 118 is shown. The first set of collagen 118 has an average collagen alignment direction 120. The average collagen alignment direction 120 is the general average direction in which collagen 118 in the relevant region (e.g., the first region 116) is aligned. As shown, the incoming ultrasonic waves 110 impinge the first set of collagen 118 in a direction that is generally perpendicular to the average collagen alignment direction 120. In other words, the angle of insonification is generally at ninety degrees in the first region 116, where the angle of insonification is the angle between the impinging ultrasonic waves 110 and the collagen alignment direction 120 in the first region 116. After impingement, backscatter 122 then reflects or arises from the first region 116. Generally, backscatter is greatest where the angle of insonification is ninety degrees.

FIG. 1D includes an exemplary representation 123 of ultrasonic waves 110 impinging a second region 124 having a second set of collagen 126. An average collagen alignment direction 128 in this second region 124 is generally parallel to the impinging waves 110. In other words, the incoming ultrasonic waves 110 impinge the second set of collagen 126 in a direction that is generally parallel to the average collagen alignment direction 128 in the region 124. To put yet another way, the angle of insonification is generally at one hundred eighty degrees to the average collagen alignment direction 128 in the second region 124, where the angle of insonification is the angle between the impinging ultrasonic waves 110 and the collagen alignment direction 128 in the second region 124. Generally, backscatter 129 from this region 124 is at a minimum. As cross-linking between collagen fibers further decreases, backscatter may increase in these parallel regions (e.g., the second region 124) since reduction in cross-linking may change the average collagen alignment direction 128.

Referring back to FIG. 1A, the average collagen alignment direction of the organ 112 is represented by dashed lines 130. In the organ 112 shown (i.e., the heart 112), the average collagen alignment direction varies as one circumnavigates the heart 112. In other examples of organs or tissue, however, the average collagen alignment direction may not vary, vary very little, or vary significantly.

In the exemplary heart 112 shown in FIG. 1A, the average collagen alignment direction 120 in the first region 116 is generally perpendicular to the to the incoming ultrasonic waves 110 impinging on the first region 116. In other words, a first angle of insonification 132 is substantially ninety (90) degrees such that incoming ultrasonic waves 110 are generally perpendicular 134 to the average collagen alignment direction 120 at the first region 116. At the second region 124, however, a second angle of insonification 136 is substantially one hundred eighty (180) degrees such that incoming ultrasonic waves 110 are substantially parallel 138 to the average collagen alignment direction 128 in this second region 124. It is noted that while the first angle of insonification 132 is ninety degrees and the second angle of insonification 136 is one hundred eight degrees in FIG. 1A and B, other angles of insonification could instead be employed.

With continued reference to FIG. 1A, the transducer 106 is configured to receive ultrasonic wave backscatter from the heart 112. The system 200 (e.g., the compute system 108) quantifies ultrasonic backscatter 140 received from the heart 112 via the first position 116 and ultrasonic backscatter 142 received from the heart 112 via the second position 124. A difference (a.k.a. a first difference or first backscatter difference) between the first quantity of backscatter 140 and the second quantity of backscatter 142 may then be determined by the compute system 108 (e.g., by at least one processor of the compute system 108).

The first difference may be used to assess the health of the organ or tissue 112. For example, the first difference could be compared to reference data (e.g., a reference table), where the reference data includes organ or tissue data associated with a healthy organ and/or tissue. Based on comparison of the reference data with the first difference, relative health of the organ or tissue 112 may be determined. Effectively, the first difference serves as a health characteristic.

Alternatively, the first difference may be saved (e.g., by one or more stage mediums of the compute system 108 or other system) to be compared to assessment data taken from the organ 112 at a later time. For example, and with reference now to FIG. 1B, additional ultrasonic data may be collected during the second session 104. That is, ultrasonic backscatter 144 received from the first region 116 and ultrasonic backscatter 146 received from the second region 124 may be again quantified. As such, a third quantity of backscatter 144 from the first region 116 may be identified or obtained and a fourth quantity of backscatter 146 received from the second region 124 may be identified or obtained by the compute system 108 (or one or more storage mediums thereof).

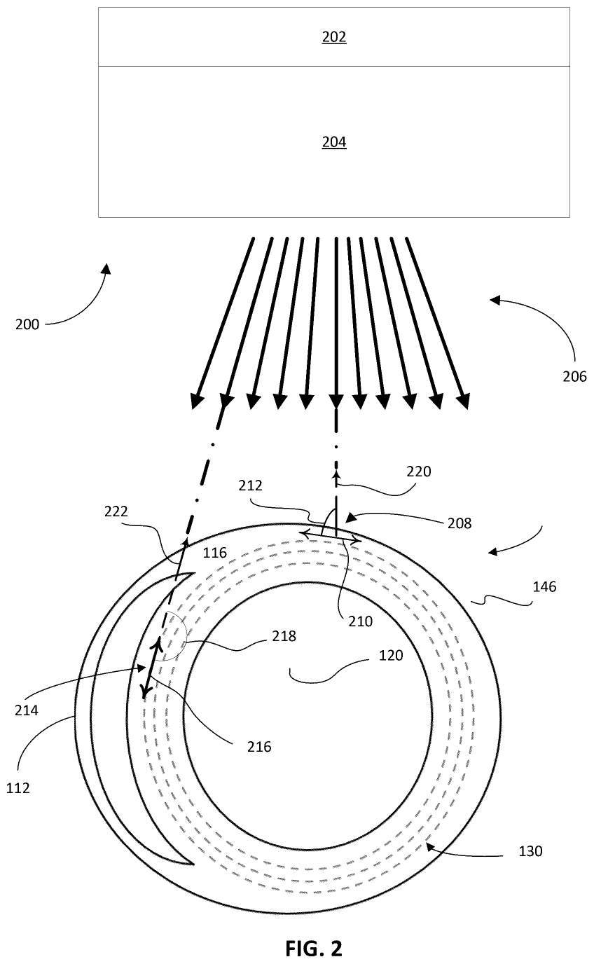

Similar to the backscatter quantification discussed above with respect to the first session 102, the third and fourth quantities of backscatter 144, 146 may be acquired via a single ultrasound acquisition or by two ultrasound acquisitions (see, e.g., FIGS. 3A-3B) during the second session 104.

It is noted that unless stated otherwise, the terms “first”, “second”, “third”, and “fourth” are merely employed to distinguish different quantities and do not imply a magnitude or order. As such, the first quantity of backscatter 140 refers to backscatter received from the first region 116 during the first session 102, the second quantity of backscatter 142 refers to backscatter received from the second region 124 during the first session 102, the third quantity of backscatter 144 refers to backscatter received from the first region 116 during the second session 104, and the fourth quantity of backscatter 146 refers to backscatter received from the second region 124 during the second session 104.

Further, the first region 116 referenced during the first session 102 and the first region 116 referenced during the second session 104 need to be at an equivalent region as the first session. Since ultrasound acquisition is being repeated at a later time (compare first session 102 to second session 104), there may be errors in alignment. The same components or regions of the organ should be measured. Misalignment, for example, relative to the ultrasound wave 110 of the region 116 during the second session by 10 degrees in either direction may result approximately 5% error in the quantity of backscatter 144.

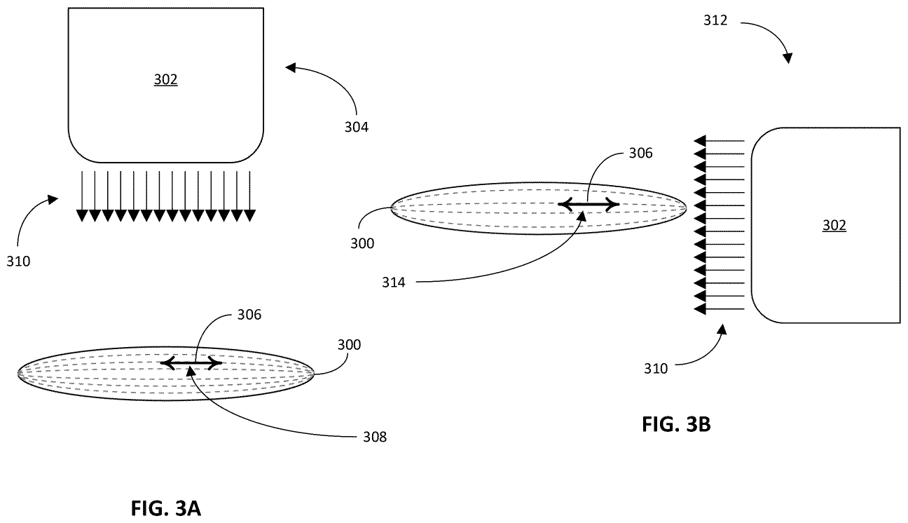

With continued reference to FIG. 1B, after the third and fourth quantities of backscatter 144, 146 are quantified, a difference (a.k.a. a second difference) between the first quantity 144 and the second quantity 146 may be determined. The second difference (e.g., an absolute or an absolute mean difference between the third and fourth quantities of backscatter 144, 146) may then be compared to the first difference determined from the first session 102 (see, e.g., FIG. 4). Accordingly, any change over time (i.e., the time from the first session 102 to the second session 104) may be identified. Accordingly, this change, or lack thereof, may be employed to assess the health of the organ or tissue 112. Like the first difference, the second difference also serves as a health characteristic.

If, for example, the second difference from the second session 104 is less than the first difference from the first session 102, then fibrosis or fibrotic crosslinking may have decreased. If the difference from the second session 104 is greater than the difference from the first session 102, fibrosis or fibrotic crosslinking may have increased. And if the difference from the second session 104 is equal (or substantially equal) to the difference from the first session 102, fibrosis or fibrotic crosslinking may have remained unchanged.

If a treatment were applied to the organ 112 during the intervening time between the first session 102 and the second session 104, the effectiveness of the treatment may be determined based on the difference comparisons.

It is noted that that the first position 116 and the second position 124 shown in FIGS. 1A and 1B are merely exemplary. Indeed many positions in the organ 112 may be employed.

While FIGS. 1A-1B represent a transducer 106 that conveys ultrasonic waves 110 in a parallel manner, transducers that convey ultrasonic waves in a non-parallel manner may also be employed to assess health.

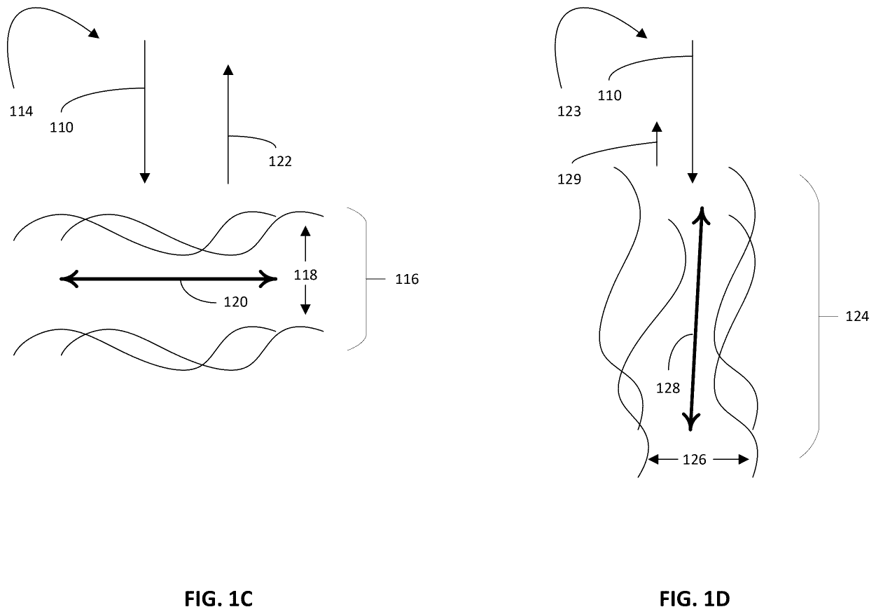

For example, FIG. 2 illustrates a system 200 that includes a compute system 202 and an ultrasonic transducer 204 that conveys ultrasonic waves 206 in a radial manner. The transducer 204 is configured to also receive ultrasonic backscatter from the organ or tissue 112.

A first region 208 if FIG. 2 is identified (e.g., via the compute system 202 or a user), where ultrasonic waves 206 intersect an average collagen alignment direction 210 of the first region 208 at a first angle of insonification 212. It is noted that in the example shown, the first angle of insonification 210 is not quite ninety degrees.

A second region 214 is also identified (e.g., via the compute system 202 or a user), where ultrasonic waves 206 convey along an average collagen alignment direction 216 in the second region 214 at a second angle of insonification 218. It is noted that in the example shown, the second angle of insonification 218 is substantially one hundred eighty degrees, though it does not need to be. Nonetheless, health assessments may be more accurate the more substantially different the two angles of insonification are from each other (e.g., selecting the brightest region of an image and the darkest region of an image). It is also noted that, at times, there may be more than one region that provides maximum or near-maximum backscatter. Similarly, there may be more than one region that provides minimal, or near-minimal, backscatter. As such, the compute system 202 or user may have more than one option to select for the first region and more than one option to select for the second region.

Ultrasonic backscatter 220, 222 can be quantified in a similar manner to that discussed above with respect to FIGS. 1A-1B. Likewise difference values (a.k.a. health characteristics) between backscatter 220, 222, and difference comparisons from data received over different sessions can also be made in a similar manner to that described above with respect to FIG. 1A-1B.

As illustrated in FIG. 2, even when the incoming ultrasonic waves 206 are not uniformly parallel to one another, regions 208, 214 can still be selected such that their respective average collagen alignment directions 210, 216 are perpendicular or substantially perpendicular to maximize effectiveness.

In light of this disclosure, it is clear region locations may differ due to the manner in which the ultrasonic waves (e.g., incoming waves 110, 206) impinge the organ. That is, since the ultrasonic waves 206 of FIG. 2 are conveyed from the transducer 304 in a radial manner, the identified locations or regions 208, 214 are different than those shown in FIGS. 1A and 1B, where the ultrasonic waves 110 are conveyed in a substantially parallel manner.

Using similar techniques, other transducers that convey ultrasonic waves in different geometries may also be employed. The system merely needs to identify locations where ultrasonic impingement on collagen alignment is substantially parallel and substantially perpendicular to the conveyed ultrasonic waves. As is clear from this disclosure, the locations may be dependent on the manner in which the transducer conveys the impinging ultrasonic waves.

While the discussion above uses the heart as an exemplary organ, techniques and systems discussed herein may be employed on other organs having different collagen alignment characteristics. Take, for example, FIGS. 3A and 3B, where collagen alignment in an organ 300 does not substantially change throughout the organ 300. With a transducer 302 in a first orientation 304 to the organ 300 in FIG. 3A, an average collagen alignment direction 306 in a first region 308 is generally perpendicular to impinging ultrasonic waves 310 coming from an ultrasonic transducer 302.

Since collagen alignment does not substantially change throughout the organ 300, it may be difficult to select or identify a second region wherein a collogen alignment direction is substantially parallel to the incoming ultrasonic waves 310. To rectify, the first orientation 304 between organ 300 and the transducer 302 may be changed to a second orientation 312, as shown in FIG. 3B.

As such, a second region 314 can be selected or identified such that the average collagen alignment direction 306 of the second region 314 is substantially parallel to the incoming waves 310, thus maximizing the effectiveness of assessments. In such a scenario, one ultrasound acquisition may be carried while in the first orientation 304, and another ultrasound acquisition may be carried out while in the second orientation 312. Backscatter differences (health characteristics) may, therefore, be determined via two ultrasound acquisitions.

FIGS. 3A and 3B also illustrate that there are examples where the first region 308 and the second region 314 may be substantially the same regions.

Referring now to FIG. 4, an exemplary graph 400 for visually assessing health of a tissue or organ over time is shown. A first backscatter difference 402 (a health characteristics) and a second backscatter difference 404 (a health characteristics) are plotted on the exemplary graph 400. The first backscatter difference 402 represents the absolute mean difference between backscatter data collected during a first session 406, while the second backscatter difference 404 represents the absolute mean difference between backscatter data collected during a second session 408. For example, and with reference to FIGS. 4 and 1A-1B, the first backscatter difference 402 may represent the absolute value of the difference between the backscatter 140 arising from the first region 116 and the backscatter 142 arising from the second region 124 during the first session 102, 406. Similarly, the second backscatter difference 404 may represent the absolute value of the difference between the backscatter 144 arising from the first region 116 and the backscatter 146 arising from the second region 124 during the second session 104, 408.

Since the value of the second backscatter difference 404 is lower than the value of the first backscatter difference 402, it may be inferred that fibrotic cross-linking has decreased in the organ 112 between the time of the first session 102, 406 and the second session 104, 408. Such reduction in in fibrotic cross-linking may have caused an increase in the backscatter 146 arising from the second region 124 during the second session 104, 408. That is, a reduction in fibrotic cross-linking after the first session 102, 406 may cause an increase in backscatter 146 in regions where the average collagen alignment direction 128 is parallel or substantially parallel to the incoming ultrasonic waves 110.

A treatment, for example, may have been prescribed, thus causing the decrease from the first backscatter difference 402 to the second backscatter difference 404. It is noted, a reduction in fibrotic cross-linking in an organ or tissue is not always beneficial. Depending on the circumstance, it may be detrimental. Nonetheless, evaluating backscatter difference values over time may been helpful for assessing the state of health of the tissue or organ.

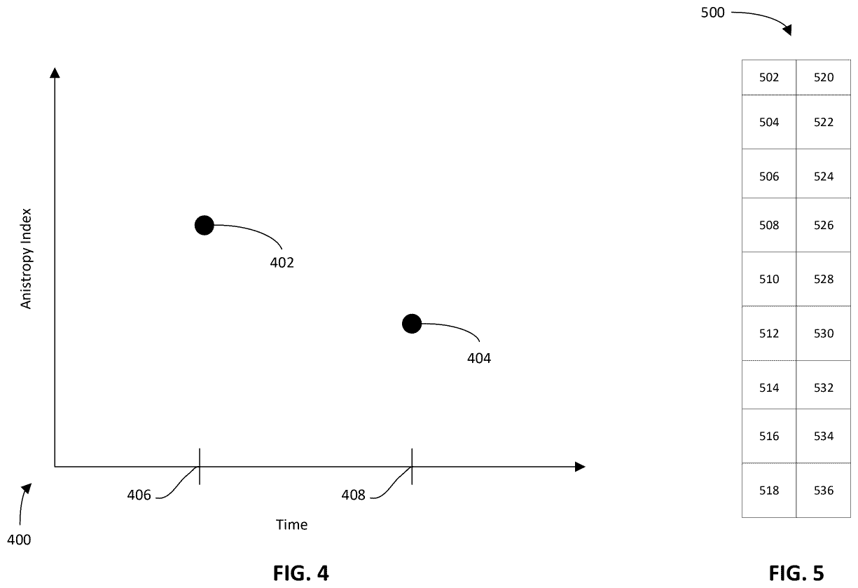

Evaluating an individual backscatter difference value may also be beneficial. For example, and with reference to FIG. 5, an exemplary reference table 500 is shown. The exemplary reference table 500 includes a first column 502 representing a different organs or tissues in each row. Eight different organs and/or tissues 504-518 are represented in the first column 502. Under a second column 520, representative healthy backscatter difference values 522-536 are represented.

A backscatter difference value (e.g., first or second difference 402, 404 of FIG. 4) may be compared to the reference table 500. That is, once a backscatter difference value is determined, the corresponding tissue or organ 504-518 can be identified under the first column 502. Then, backscatter difference value can be compared to the corresponding healthy value 522-536 under column 2 520. In other words, the reference chart allows the computed backscatter difference value to be compared to a known healthy value 522-536. Such a comparison can be employed to assess the health of the organ or tissue in question.

It is noted that techniques described herein could be applied retroactively. That is, already obtained ultrasonic images or data could be employed. In other words, a compute system having instruction stored thereon, or access thereto, could gather relevant data from previously obtained images or data and makes first region selections and second region selections therefrom to employ the relevant backscatter data to assess the health of a tissue or organ in the manner set forth above with respect to FIGS. 1A-5.

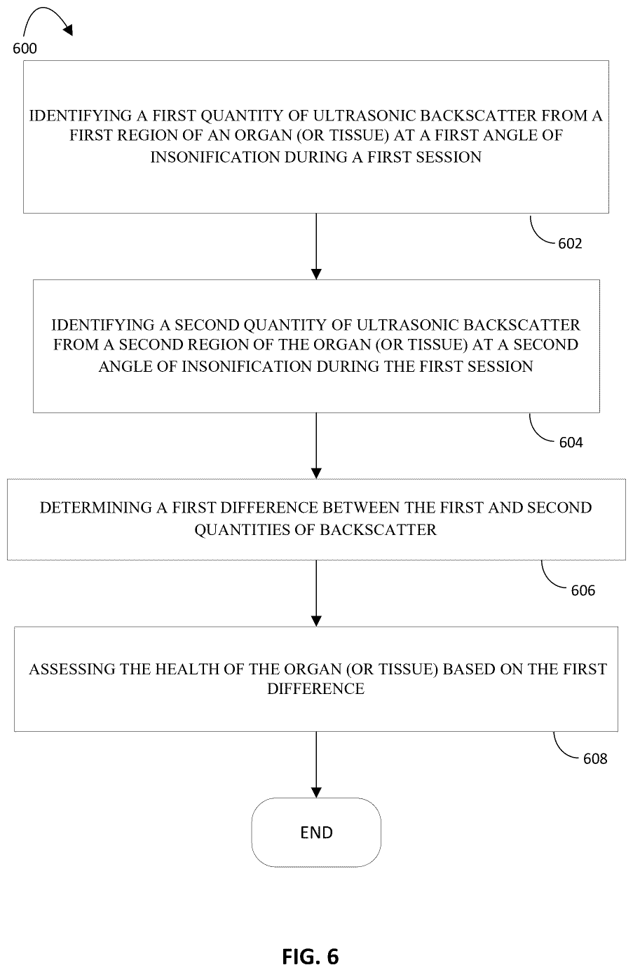

With reference now to FIG. 6, an exemplary technique 600 for assessing health of a tissue or organ is shown. At block 602, identifying a first quantity of ultrasonic backscatter from a first region of an organ (or tissue) at a first angle of insonification during a first session (e.g., an ultrasonic session or ultrasonic imaging session) occurs. The identification of the first quantity of ultrasonic backscatter may be associated with an ultrasound acquisition (i.e., data acquisition via an ultrasound transducer) during the first session.

In one example, the first angle of insonification is perpendicular or generally perpendicular to an average collagen alignment direction in the first region of the organ or tissue. Other angles, however, are envisioned.

At block 604, identifying a second quantity of ultrasonic backscatter from a second region of the organ (or tissue) at a second angle of insonification during the first session occurs. In one example, the second angle of insonification is parallel or generally parallel to an average collagen alignment direction in the second region of the tissue or organ. Other angles of insonification, however, may be employed.

The identification of the second quantity of ultrasonic backscatter may be associated with another ultrasound acquisition during the first session. In other words, during the first session one ultrasound acquisition may employed for identifying the first quantity and another ultrasound acquisition may be employed for identifying the second quantity.

Alternatively, identification of the first and second quantities of ultrasonic backscatter may each be associated with one ultrasound acquisition carried out during the first session. For example, one ultrasound acquisition may be employed to gather backscatter data from the organ or tissue during the first session. The first and second quantities of backscatter may then be identified in that backscatter data.

With continued reference to FIG. 6, at block 606, determining a difference (a.k.a. a first difference) between the first and second quantities of backscatter occurs. The determination of the difference (e.g., an absolute or an absolute mean difference) may be carried out by, for example, a compute system or processor thereof.

After determining the first backscatter difference, process control proceeds to block 608 and assessing the health of the organ or tissue based on the first difference is carried out. For example, the first difference may be compared to a reference index, where the reference index lists values associated with healthy organs or tissues. By comparing the first difference to the reference index, an assessment of the organ or tissue health can be carried out. See FIGS. 4 and 5 for further examples of how the health of an organ or tissue may be assess using the backscatter differences.

Once the assessment is made at block 608 of FIG. 6, process control proceeds to an end.

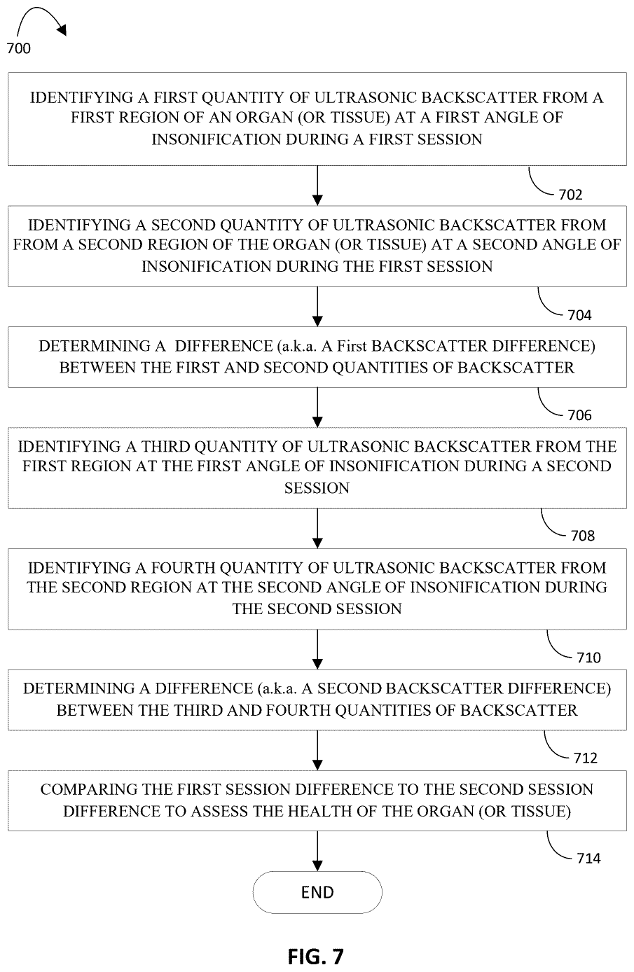

Referring to FIG. 7, a technique 700 for assessing the health of a tissue or organ over time is shown. At block 702, identifying a first quantity of ultrasonic backscatter from a first region of an organ (or tissue) at a first angle of insonification during a first session (e.g., an ultrasonic session or ultrasonic imaging session) occurs. The identification of the first quantity of ultrasonic backscatter may be associated with an ultrasound acquisition (i.e., data acquisition via an ultrasound transducer) during the first session.

In one example, the first angle of insonification is perpendicular or generally perpendicular to an average collagen alignment direction in the first region of the organ or tissue. Other angles, however, are envisioned.

At block 704, identifying a second quantity of ultrasonic backscatter from a second region of the organ (or tissue) at a second angle of insonification during the first session occurs. In one example, the second angle of insonification is parallel or generally parallel to an average collagen alignment direction in the second region of the tissue or organ. Other angles of insonification, however, may be employed.

The identification of the second quantity of ultrasonic backscatter may be associated with another ultrasound acquisition during the first session. In other words, during the first session one ultrasound acquisition may employed for identifying the first quantity and another ultrasound acquisition may be employed for identifying the second quantity.

Alternatively, identification of the first and second quantities of ultrasonic backscatter may each be associated with one ultrasound acquisition carried out during the first session. For example, one ultrasound acquisition may be employed to gather backscatter data from the organ or tissue during the first session. The first and second quantities of backscatter may then be identified in that backscatter data. Further details regarding ultrasound acquisition(s) will be set forth above with respect to FIG. 1A-6.

With continued reference to FIG. 7, at block 706, determining a difference (a.k.a. a first difference) between the first and second quantities of backscatter occurs. The determination of the difference (e.g., an absolute or an absolute mean difference) may be carried out by, for example, a compute system or processor thereof.

Proceeding to block 708, identifying a third quantity of ultrasonic backscatter from the first region (or at least partially from the first region) at the first angle (or at least substantially the first angle) of insonification during a second session is carried out. This second session occurs after the first session. The second session may occur second(s), day(s), week(s), month(s), or year(s) after the first session.

The first session could, for example, occur during a doctor's visit (e.g., a checkup). In such an example, the second session may then occur during another visit (e.g., the following year's checkup). As yet another example, the first session could be where a baseline of the organ or tissue is gathered prior to applying a treatment to the tissue or organ, while the second session could be where treatment of the organ or tissue is evaluated via techniques described herein.

Identifying a fourth quantity of ultrasonic backscatter from the second region (or at least partially from the second region) at the second angle (or at least substantially the first angle) of insonification during the second session is carried out at block 710. Similar to identification of the first and second quantities (see e.g., blocks 702 and 704 with accompanying descriptions), one or two ultrasound acquisitions could be carried out during the second session to gather the data associated with the third and fourth quantities of backscatter.

Proceeding to block 712, process control carries out determining a difference (a.k.a. a second difference or second backscatter difference) between the third and fourth quantities of backscatter. The second backscatter difference may be an absolute value of the difference between the relevant backscatter quantities (see blocks 708-710).

After the second backscatter difference is determined, process control proceeds to block 714, where comparing the first backscatter difference to the second backscatter difference to assess the health of the organ (or tissue) is carried out. See FIGS. 4 and 5 for some examples of how the health of an organ or tissue may be assess using the backscatter differences.

After making the comparison at block 714 of FIG. 7, process control comes to an end.

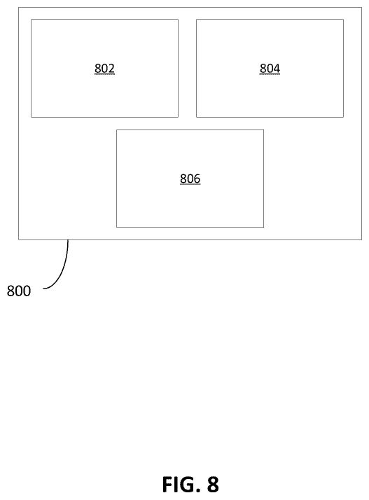

With reference now to FIG. 8, an exemplary compute system 800 is shown. The exemplary compute system 800 may be substituted for the compute systems discussed above (e.g., compute system 108, FIGS. 1A and 1B and compute system 202, FIG. 2). The compute system 800 may include at least one computer readable storage medium 802 (memory) having instructions thereon to carry out the techniques and etc., at least one processor 804 to make calculations and etc., and/or a user interface 806 (e.g., a screen and/or keyboard).

FIGS. 1A-8 discussed above described systems and/or techniques for assessing health of an organ or tissue. The discussion below describes study results.

It should be understood that a compute device or system (e.g., computing device, remote computing device, user device, etc.), a system, and/or a processor as described herein may include a conventional processing apparatus known in the art, which may be capable of executing preprogrammed instructions stored in an associated memory, all performing in accordance with the functionality described herein. To the extent that the methods described herein are embodied in software, the resulting software can be stored in an associated memory and can also constitute means for performing such methods. Such a system or processor may further be of the type having ROM, RAM, RAM and ROM, and/or a combination of non-volatile and volatile memory so that any software may be stored and yet allow storage and processing of dynamically produced data and/or signals.

It should be further understood that an article of manufacture in accordance with this disclosure may include a non-transitory computer-readable storage medium having a computer program encoded thereon for implementing logic and other functionality described herein. The computer program may include code to perform one or more of the methods disclosed herein. Such embodiments may be configured to execute via one or more processors, such as multiple processors that are integrated into a single system or are distributed over and connected together through a communications network, and the communications network may be wired and/or wireless.

Computing devices (e.g., computing device, remote computing device, user device, etc.) may include any computing device such as a computer, mobile device, cellular phone, smartphone, smartwatch, activity tracker, tablet computer, next generation portable device, handheld computer, notebook, laptop, projector device (e.g., three-dimensional holographic or hologram projector), or virtual reality or augmented reality device. The devices may include a transceiver that communicates information between any of devices, a server, and a database.

A server may include any computing system. A server may communicatively connect with and transfer information with respect to the devices and a database. The server may be in continuous or periodic communication with the devices and a database. The server may include a local, remote, or cloud-based server or a combination thereof and may be in communication with and provide information to any or a combination of the devices. Server may further provide a web-based user interface (e.g., an internet portal) to be displayed by the user interface.

The devices (e.g., computing device, remote computing device, user device, etc.) may be connected via any wired or wireless connections between two or more endpoints, for example, to facilitate transfer of information. Connection may include a local area network, for example, to communicatively connect the devices, with a network. Connection may include a wide area network connection, for example, to communicatively connect a server with a network. Connection may include a wireless connection, e.g., radiofrequency (RF), near field communication (NFC), Bluetooth communication, Wi-Fi, or a wired connection, for example, to communicatively connect the devices.

While the disclosed materials have been described in detail in connection with only a limited number of embodiments, it should be readily understood that the embodiments are not limited to such disclosed embodiments. Rather, that disclosed can be modified to incorporate any number of variations, alterations, substitutions or equivalent arrangements not heretofore described, but which are commensurate with the spirit and scope of the disclosed materials. Additionally, while various embodiments have been described, it is to be understood that disclosed aspects may include only some of the described embodiments. Accordingly, that disclosed is not to be seen as limited by the foregoing description, but is only limited by the scope of the appended claims.

What is claimed is:

1. A compute system configured to:
store, on at least one computer readable storage medium of the compute system, a first quantity associated with ultrasonic backscatter arising from a first region of an organ at a first angle of insonification during a first session;
store, on the at least one computer readable storage medium, a second quantity associated with ultrasonic backscatter arising from a second region of the organ at a second angle of insonification during the first session;
determine, via at least one processor of the compute system, a first difference between the first quantity and the second quantity; and
provide a health characteristic of the organ to a user interface, wherein the health characteristic is based on the first difference.

2. The compute system of claim 1, wherein the first angle of insonification is an angle between a first average collagen alignment direction in the first region and incoming ultrasonic wave direction in the first region, and wherein the second angle of insonification is an angle between a second average collagen alignment direction in the second region and incoming ultrasonic wave direction in the second region.

3. The compute system of claim 1 further configured to:
store, on at least one of the at least one computer readable storage medium, a third quantity associated with ultrasonic backscatter arising from the first region of the organ during a second session;
store, on at least one of the at least one computer readable storage medium, a fourth quantity associated with ultrasonic backscatter arising from the second region of the organ during the second session; and
determine, via at least one of the at least one processor of the compute system, a second difference between the third quantity and the fourth quantity.

4. The compute system of claim 3 further configured to compare the first difference with the second difference, wherein the health characteristic is further based on the comparison of the first difference with the second difference.

5. The compute system of claim 2, wherein the first angle of insonification is substantially perpendicular to the first average collagen alignment direction in the first region, and wherein the second angle of insonification is substantially parallel to the second average collagen alignment direction in the second region.

6. The compute system of claim 3, wherein the second session occurs at a later time than the first session.

7. The computes system of claim 3, wherein the first region is at least partially the same as the second region, and wherein the first quantity is associated with a first ultrasound image acquisition where an ultrasound transducer was at a first orientation to the organ, and wherein the second quantity is associated with a second ultrasound image acquisition where the ultrasound transducer was at a second orientation to the organ.

8. A system comprising:
at least one ultrasonic transducer configured to convey ultrasonic waves to an organ;
a compute system comprising at least one computer readable storage medium having instructions thereon that:
identify a first quantity of ultrasonic backscatter from a first region of an organ at a first angle of insonification during a first session;
identify a second quantity of ultrasonic backscatter from a second region of the organ at a second angle of insonification during the first session, wherein the first quantity of ultrasonic backscatter and the second quantity of ultrasonic backscatter arises from reflection of the ultrasonic waves;
determine a first difference between the first quantity of ultrasonic backscatter and the second quantity of ultrasonic backscatter; and
provide a health characteristic of the organ to a user, wherein the health characteristic is based on the first difference.

9. The system of claim 8, wherein the first angle of insonification is an angle between a first region average collagen alignment direction and first region incoming ultrasonic wave direction, and wherein the second angle of insonification is an angle between a second region collagen alignment direction and second region incoming ultrasonic wave direction.

10. The system of claim 9, wherein the at least one computer readable storage medium has further instructions to:
identify a third quantity of ultrasonic backscatter from substantially the first region of the organ at substantially the first angle of insonification during a second session, wherein the second session is at a later time than the first session;
identify a fourth quantity of ultrasonic backscatter from substantially the second region of the organ at substantially the second angle of insonification during the second session; and
determine a second difference between the third quantity of ultrasonic backscatter and the fourth quantity of ultrasonic backscatter, wherein the health characteristic is further based on the second difference.

11. The system of claim 10, wherein the first angle of insonification is substantially perpendicular to the first region average collagen alignment direction, and wherein the second angle of collagen alignment is substantially parallel to a second region collagen alignment direction.

12. The system of claim 8, wherein the first region is substantially at a different location as the second region, and wherein the ultrasonic backscatter from the first region arises from a first ultrasonic acquisition and ultrasonic backscatter from the second region also arises from the first ultrasonic acquisition.

13. The system of claim 8, wherein the first region is substantially at a same location as the second region, and wherein ultrasonic backscatter from the first region arises from a first ultrasonic acquisition and ultrasonic backscatter from the second region arises from a different ultrasonic acquisition.

14. The system of claim 10, wherein the at least one computer readable storage medium has further instructions to compare the first difference with the second difference.

15. A method comprising:
identifying a first quantity of ultrasonic backscatter from a first region of an organ at a first angle of insonification during a first session;
identifying a second quantity of ultrasonic backscatter from a second region of the organ at a second angle of insonification during the first session;
determining, via at least one compute processor, a first difference between the first quantity of ultrasonic backscatter and the second quantity of ultrasonic backscatter; and
assessing health of the organ based on the first difference.

16. The method of claim 15, wherein the first angle of insonification is an angle between collagen alignment direction in the first region and incoming ultrasonic wave direction in the first region, and wherein the second angle of insonification is an angle between collagen alignment direction in the second region and incoming ultrasonic wave direction in the second region.

17. The method of claim 16 further comprising:
identifying a third quantity of ultrasonic backscatter from substantially the first region of the organ at substantially the first angle of insonification during a second session, wherein the second session is at a later time than the first session;
identifying a fourth quantity of ultrasonic backscatter from substantially the second region of the organ at substantially the second angle of insonification during the second session; and
determining a second difference between the third quantity of ultrasonic backscatter and the fourth quantity of ultrasonic backscatter, wherein assessing health of the organ is further based on the second difference.

18. The method of claim 15, wherein the first angle of insonification is substantially perpendicular to collagen alignment direction in the first region, and wherein the second angle of collagen alignment is substantially parallel to collagen alignment direction in the second region.

19. The method of claim 15, wherein the first region is substantially at a different location as the second region, and wherein ultrasonic backscatter from the first region arises from a first ultrasonic acquisition and ultrasonic backscatter from the second region also arises from the first ultrasonic acquisition 20. The method of claim 15, wherein the first region is substantially at a same location as the second region, and wherein ultrasonic backscatter from the first region arises from a first ultrasonic acquisition and ultrasonic backscatter from the second region arises from a different ultrasonic acquisition.

21. The method of claim 15, wherein assessing health of the organ is further based on comparing the index to reference data associated with average organ health characteristics.

22. The method of claim 17, wherein assessing health of the organ comprises comparing the first difference from the first session with the second difference from the second session.
